(19) United States
(12) Patent Application Publication
(10) Pub. No.: US 2024/0358621 A1
(43) Pub. Date: Oct. 31, 2024

(54) PERSONAL CARE COMPOSITION

(71) Applicant: Conopco, Inc., d/b/a UNILEVER, Englewood Cliffs, NJ (US)

(72) Inventors: James Michael BIANCHI, Wallingford, CT (US); Heather Xiaolan Liu, San Francisco, CA (US)

(21) Appl. No.: 18/139,740

(22) Filed: Apr. 26, 2023

(51) Int. Cl. A61K 8/891; A61K 8/02; A61K 8/26; A61K 8/28; A61K 8/34; A61K 8/86; A61K 8/92; A61Q 15/00

(52) U.S. Cl. CPC A61K 8/891; A61K 8/0229; A61K 8/26; A61K 8/28; A61K 8/342; A61K 8/86; A61K 8/922; A61Q 15/00; A61K 2800/31

(57) ABSTRACT

Disclosed herein is a personal care composition. In particular, disclosed herein is a substantially anhydrous personal care composition comprising at least one non-volatile emollient oil having a boiling point of at least 215° C. at atmospheric pressure, a lipophilic material, and less than 25% antiperspirant active by weight of the composition, wherein the composition has reduced volatile silicones, and further wherein the composition is substantially free of emulsifier. The disclosed personal care composition is especially useful as an efficacious antiperspirant or deodorant stick with reduced volatile silicone content that provides parity payout and sensory benefits upon topical application to skin.

### FIELD OF THE INVENTION

Disclosed herein is a personal care composition. In particular, disclosed herein is a substantially anhydrous personal care composition comprising at least one non-volatile emollient oil having a boiling point of at least 215° C. at atmospheric pressure, a lipophilic material, and less than 25% antiperspirant active by weight of the composition, wherein the composition has reduced volatile silicones, and further wherein the composition is substantially free of emulsifier. The disclosed personal care composition is especially useful as an efficacious antiperspirant or deodorant stick with reduced volatile silicone content that provides parity payout and sensory benefits upon topical application to skin.

### BACKGROUND OF THE INVENTION

Perspiration, an innate means of thermoregulation in humans, is the production of sweat, which is a fluid secretion containing a mixture of protein, lipid, and other organic compounds. This fluid is initially odorless until bacteria on the skin metabolize and decompose the sweat, resulting in malodorous sweat by-products.

Personal care compositions, such as antiperspirants and deodorants, used to provide wetness and/or malodor protection benefit upon topical application to desired surfaces of the human body, namely the underarm regions, viz., the axilla, often comprise an astringent metal salt as antiperspirant active, notably aluminum or zirconium salt.

Many such compositions comprise substantial amounts of silicone to provide for an enhanced, silky sensory experience. There is increasing consumer and industry interest in developing antiperspirant and deodorant compositions with reduced amounts of volatile silicone without sacrificing efficacy by means of payout and hardness, as well as the sensory benefit experienced upon topical application to a surface, preferably, human skin.

The composition disclosed herein is particularly concerned with substantially anhydrous personal care compositions comprising at least one non-volatile emollient oil having a boiling point of at least 215° C. at atmospheric pressure, 1 to 30% a lipophilic material by weight of the composition, and less than 25% antiperspirant active by weight of the composition, wherein the composition has reduced volatile silicones, and further wherein the composition is substantially free of emulsifier.

### SUMMARY OF THE INVENTION

In a first aspect, there is provided a personal care composition comprising at least one non-volatile emollient oil having a boiling point of at least 215° C. at atmospheric pressure, 1 to 30%, or 5 to 25% a lipophilic material by weight of the composition, and less than 25% antiperspirant active by weight of the composition, wherein the composition has reduced volatile silicones, preferably, volatile cyclic polydimethylsiloxane, more preferably, volatile cyclic polydimethylsiloxane having three to seven silicon atoms, and further wherein the composition is substantially free of emulsifier.

In a second aspect, the personal care composition disclosed is a substantially anhydrous, preferably, oil-continuous, and, more preferably, anhydrous, antiperspirant or deodorant composition.

In a third aspect, the disclosed personal care composition provides for a solid, roll-on, or cream product comprising the composition of the first aspect comprised within appropriate packaging formats, including a stick dispenser product package, roll-on dispenser product package, or a jar. It is preferable that the composition is a solid product comprised within a stick dispenser product package.

In a fourth aspect, there is provided a method of attaining antiperspirant and/or deodorant benefits comprising the steps of: (a) identifying skin in need of antiperspirancy and/or deodorant, preferably, the axillae; and (b) topically applying, directly or indirectly, to the skin a composition made according to the first aspect of the invention disclosed herein.

In a fifth aspect, there is provided a use of a personal care composition according to the first aspect of the invention for delivering an antiperspirant and/or deodorant benefit.

In a sixth aspect, there is provided a method of manufacturing the personal care composition according to the first aspect of the invention.

All other aspects of the disclosed personal care composition will become more readily apparent upon considering the detailed description, data, and examples which follow.

Unless explicitly stated otherwise, all ranges described herein are meant to include all ranges subsumed therein. Except in the operating and comparative examples, or where otherwise explicitly indicated, all numbers used in this description indicating amounts, or ratios of materials and/or use thereof are to be understood as modified by the word “about.”

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated.

### DETAILED DESCRIPTION OF THE INVENTION

The personal care compositions disclosed herein have reduced volatile silicones concentrations. As used herein, the term “volatile” refers to a material having a boiling point of lower than 215° C. at atmospheric pressure. “Reduced volatile silicone(s)”, as used herein, is intended to mean comprising less than 30%, preferably, less than 25%, more preferably, less than 20% volatile silicone by weight of the composition. In one embodiment, the silicone is a volatile cyclic polydimethylsiloxane (i.e., cyclomethicone), preferably having three to seven silicon atoms, or having four to six silicon atoms. In another embodiment, the silicone is cyclotetrasiloxane (D4), cyclopentasiloxane (D5), cyclohexasiloxane (D6), or mixtures thereof. In still another embodiment, the silicone is cyclopentasiloxane. The disclosed personal care composition comprises no less than 5%, or 5 to 30%, or 6 to 25%, or 7 to 20%, or 8 to 17%, or 9 to 17%, or 10 to 16%, or 10 to 14.5%, or 12 to 15.5%, or 12 to 15% by weight total volatile silicone in the composition. In another embodiment, the composition comprises less than 2%, or less than 1% by weight cyclotetrasiloxane based on the total weight of the composition. In yet another embodiment, the composition comprises less than 5%, or less than 4% by weight cyclohexasiloxane based on the total weight of the composition.

The personal care compositions disclosed herein comprise a cosmetically acceptable carrier. The cosmetically acceptable carrier comprises at least one emollient, preferably, a non-volatile emollient oil. As used herein, the term “non-volatile” refers to an ingredient having a boiling point of at least 215° C. at atmospheric pressure. Illustrative but nonlimiting examples of suitable emollients include mineral oil, PPG-14 butyl ether, isopropyl isostearate, isostearyl isostearate, isopropyl myristate, isopropyl palmitate, petrolatum, butyl stearate, cetyl octanoate, butyl myristate, myristyl myristate, C₁₂₋₁₅ alkylbenzoate, octyldodecanol, isostearyl isostearate, octododecyl benzoate, isostearyl lactate, isostearyl palmitate, isobutyl stearate, dimethicone (e.g., 50 centistoke (cSt), 100 cSt), dimethicone copolyol, PEG-12 dimethicone, polydecene, and combinations thereof. In a preferred aspect, the at least one emollient comprises mineral oil, PPG-14 butyl ether, isopropyl palmitate, C₁₂₋₁₅ alkylbenzoate, dimethicone, isopropyl isostearate, isostearyl isostearate, or mixtures thereof. In another aspect, the at least one emollient comprises non-volatile silicones, including polyalkyl siloxanes (such as polydimethyl siloxanes), polyalkylaryl siloxanes and polyether siloxane copolymers. In still another aspect, the at least one emollient has a flash point of at least 80° C., or at least 85° C., or at least 90° C., or at least 95° C., or ranging from 80° C. to 300° C., or 85° C. to 290° C., or 90° C. to 280° C., or 95° C. to 270° C. In still another aspect, the at least one emollient has a boiling point of at least 215° C., or at least 225° C., or at least 250° C., or at least 260° C., or ranging from 215° C. to 400° C., or 225° C. to 395° C., or 250° C. to 390° C., or 260° C. to 385° C. In further still another aspect, the at least one emollient has a refractive index of at least 1.35, or at least 1.36, or from 1.35 to 1.55, or from 1.36 to 1.52, or from 1.37 to 1.50. The disclosed personal care composition comprises 15 to 45%, or 20 to 42%, or 25 to 40%, or 27 to 38% by total weight non-volatile emollient oil of the composition.

Personal care compositions as disclosed herein are substantially anhydrous. “Substantially anhydrous,” as used herein, refers to compositions having less than 5% by weight water, preferably, less than 1%, more preferably, less than 0.5%, still more preferably, less than 0.1%, and, most preferably, 0% by weight water. The compositions may be oil continuous. In a preferred embodiment, the personal care compositions disclosed herein are anhydrous, being substantially free of water, preferably, comprising no (i.e., 0% by weight) water. As used herein, “substantially free of water” means compositions having less than 5% by weight water, preferably, less than 1%, more preferably, less than 0.5%, still more preferably, less than 0.1%, and, most preferably, 0% by weight water.

The personal care composition comprises at least one antiperspirant active, preferably, an astringent metal salts comprising aluminum and/or zirconium. The personal care composition may comprise an astringent metal salt selected from the group consisting of aluminum chloride, aluminum hydrochloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate (ASCH), aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrex gly, aluminum zirconium tetrachlorohydrate, aluminum zirconium trichlorohydrate, aluminum zirconium trichlorohydrex gly. Many of such actives are listed in the U.S. Food and Drug Administration (FDA) antiperspirant drug products for over-the-counter human use monograph under C.F.R. Title 21, Sec. 350.10. The disclosed personal care compositions may comprise ASCH comprising water-soluble calcium salt and amino acid, in particular glycine. Such salts are preferable of enhanced activity, achieved by heating the ASCH with the water-soluble calcium salt and amino acid at sufficient temperature and for sufficient time for the antiperspirancy performance of the ASCH to be improved. Further details on this technology may be found in WO 2014/187685 by Unilever. In one aspect, the antiperspirant active is aluminum chlorohydrate, aluminum zirconium tetrachlorohydrex gly, aluminium sesquichlorohydrate of chemical formula Al₂OH_{4.4}Cl_{1.6} to Al₂OH_{4.9}Cl_{1.1} also comprising water-soluble calcium salt and amino acid, or a mixture thereof.

In one embodiment, non-aluminum and non-zirconium antiperspirant actives may optionally be present to use in place of, to augment, or to supplement the antiperspirant active. Non-aluminum or non-zirconium antiperspirant active is preferably selected from zinc-based deodorizing agents (such as zinc oxide), glycerol monolaurate plus isostearyl alcohol, chitosan or a salt thereof with a weight average molecular weight of from 250 to 650 kDa, titanium compound chelated by alkanolamine with an acid and a polyhydric alcohol, or cholic acid derivative selected from a hydroxycholic acid or a salt thereof with a multivalent metal salt.

In another embodiment, the personal care composition is free (i.e., 0%) of astringent metal salts. In yet another embodiment, the antiperspirant active used in the personal care compositions disclosed herein comprises at least 70%, or at least 75%, or at least 80%, or at least 90%, or entirely of (i.e., 100%) astringent metal salts by weight of the composition. In still another embodiment, the personal care composition comprises antiperspirant actives that are not astringent metal salts. The antiperspirant active may be incorporated in the disclosed personal care composition in amounts of less than 25%, or less than 24.5%, or less than 24%, or less than 23%, or less than 22%, or less than 21%, or less than 20%, or 0.1 to 24.5%, or 0.5 to 24%, or 1 to 23%, or 2.5 to 22%, or 5 to 21%, or 7.5 to 20% antiperspirant active by weight of the composition.

In one aspect of the disclosed personal care composition, the composition is substantially free of humectant. “Substantially free of,” as used herein, is intended to mean comprising less than 2%, preferably, less than 1% by weight, more preferably, less than 0.5% by weight, and, most preferably, 0% by weight. “Humectant,” as used herein, refers to an ingredient that attracts and binds water to draw water into the skin's layers. In another aspect, the personal care composition is substantially free of humectant selected from the group consisting of glycerin, PEG-4, or mixtures thereof. In yet another aspect, the composition optionally comprises a humectant selected from the group consisting of PEG-8, panthenol, urea, and mixtures thereof in amounts ranging from 0.01 to 6%, or 0.05 to 5.5%, or 1 to 5% by weight of the composition. In still another aspect, the composition comprises no (i.e., 0% by weight) humectant.

In a preferred embodiment, the personal care composition is a deodorant or antiperspirant.

The personal care composition is substantially free of emulsifier. In one embodiment, the composition is substantially free of nonionic emulsifier, anionic emulsifier, cationic emulsifier, and amphoteric/zwitteronic emulsifier. Illustrative but nonlimiting examples of nonionic emulsifier include steareth-2, ceteth-2, polyglyceryl-2 stearate, polyglyceryl-2-distearate, polyglyceryl-3 distearate, sorbitan palmitate, glycerol stearate, glycol stearate, sucrose polystearate, cetyl palmitate, sorbitan tristearate, sorbitan monopalmitate, diethylene glycol monostearate, glycerol monostearate, sorbitan monostearate, PEG-8 beewax, Sorbeth-20 beewax, PEG-2 stearate, glyceryl myristate, glyceryl oleate, and glyceryl stearate. Illustrative but nonlimiting examples of anionic emulsifier include sodium lauryl sulfate and sodium stearoyl glutamate, sodium cocoyl glycinate, sodium methyl cocoyl taurate, sodium cocoyl glutamate, sodium cocoyl isethionate, sodium laureth-13 carboxylate, trideceth-7 carboxylic acid, and sodium stearyl glutamate. Illustrative but nonlimiting examples of cationic emulsifier include stearamidopropyl dimethylamine, tetramethylammonium acetate, tetra butylammonium hydrogen sulfate, dodecyltrimethylammonium chloride, tetraethylammonium bromide, tetrabutyl ammonium chloride, and dimethyldioctadecylammonium bromide. Illustrative but nonlimiting examples of amphoteric/zwitteronic emulsifier include cocamidopropyl betaine, lauryl betaine, betaine citrate, sodium lauroamphoacetate, sodium hydroxymethylglycinate, (carboxymethyl) dimethyloleylammonium hydroxide, and (carboxylatomethyl)dimethyl(octadecyl) ammonium. In an embodiment, the personal care composition comprises no (i.e., 0% by weight) emulsifier.

The personal care composition comprises at least one lipophilic material. “Lipophilic material,” as used herein, means a material which tends to combine with or dissolve in lipids, oils or fats. Without being bound to theory, such lipophilic material may contribute to building stable structure of the personal care composition. It is preferred that the lipophilic material is a hydrogenated oil, a fatty alcohol (such as fatty alcohols such as cetyl alcohol, stearyl alcohol, arachidyl alcohol, behenyl alcohol, or mixtures thereof), a fatty acid (such as stearic acid, palmitic acid, 12-hydroxystearic acid, 16-hydroxyhexadecanoic acid, and derivatives and mixtures thereof), a fatty ester, a petroleum oil, a wax or a mixture thereof. The term “fatty” refers to carbon chain lengths ranging from 10 to 30 carbon atoms. Illustrative of this category are pelargonic, lauric, myristic, palmitic, stearic, isostearic, hydroxystearic, oleic, linoleic, ricinoleic, arachidic, behenic, erucic acids and combinations thereof. It is preferred that the lipophilic material is a hydrogenated oil selected from hydrogenated soybean oil, hydrogenated palm oil, hydrogenated corn oil, hydrogenated peanut oil, hydrogenated cottonseed oil, hydrogenated olive oil, hydrogenated avocado oil, hydrogenated castor oil, hydrogenated jojoba oil, or a mixture thereof. Desirable lipophilic material may also include polyethylene. Desirable waxes include petroleum-derived waxes (e.g., paraffin wax, petrolatum, microcrystalline wax), animal-derived waxes or those from animal byproducts (e.g., beeswax, spermaceti wax, Chinese wax, wool wax, shellac wax), plant-derived waxes (e.g., tribehenin wax, carnauba wax, candelilla wax, bayberry wax, jojoba wax, orange wax, rice bran wax, sunflower wax, castor wax, soy wax, rhuswax, sugarcane wax, Rosa damascena flower wax), mineral waxes (e.g., montan wax, ceresin wax, ozokerite) and synthetic derivatives of natural waxes. In one aspect, at least one lipophilic material used in accordance with the disclosed personal care composition is characterized by a melting point of at least 50° C., or from 50 to 120° C., or from 50 to 100° C., or from 52 to 95° C.

It is within the scope of the invention for the at least one lipophilic material to optionally include a plant-based oil. Illustrative but nonlimiting examples of plant-based oils include Adansonia digitata (Baobab) seed oil, Argania spinosa (Argan) kernel oil, Astrocaryum murumuru (Murumuru) butter, Astrocaryum aculeatum (Tucuma) butter, Borago officinalis (Borage) seed oil, Brassica campestris (Rapeseed) seed oil, Butyrospermum parkii (Shea) butter, Calophyllum inophyllum (Tamanu) seed oil, Camelina sativa (False Flax) seed oil, caprylic/capric triglyceride, Carthamus tinctorius (Safflower) seed oil, coco-caprylate/caprate, Cocos nucifera (Coconut) oil, Crambe abyssinica (Crambe) seed oil, Euterpe oleracea (Agai) fruit oil, Garcinia indica (Kokum) seed butter, Gossypium herbaceum (Cotton) seed oil, Helianthus annuus (Sunflower) seed oil, Hippophae rhamnoides (Sea Buckthorn) oil, jojoba esters, Limnanthes alba (Meadowfoam) seed oil, Linum usitatissimum (Linseed) seed oil, Macadamia ternifolia (Macadamia) seed oil, Mangifera indica (Mango) seed butter, Moringa oleifera (Moringa) seed oil, Oenothera biennis (Evening Primrose) oil, Olea europaea (Olive) fruit oil, Orbignya oleifera (Babassu) seed oil, Oryza sativa (Rice) germ oil, Persea gratissima (Avocado) oil, Platonia insignis (Bacuri) seed butter, Plukenetia volubilis (Sacha inchi) seed oil, Prunus amygdalus dulcis (Sweet Almond) oil, Prunus armeniaca (Apricot) kernel oil, Ricinus communis (Castor) seed oil, Rosa canina (Rosehip) seed oil, Rosa moschata (Rosehip) seed oil, Salvia hispanica (Chia) seed oil, Salvia officinalis (Sage) oil, Schinziophyton rautanenii (Mongongo) kernel oil, Sesamum indicum (Sesame) seed oil, Shorea Robusta (Sal) seed oil, Simmondsia chinensis (Jojoba) seed oil, soy lecithin, squalane, Theobroma Cacao (Cocoa) butter, Theobroma grandiflorum (Capuagu) seed butter, Triolein (algae oil), Triticum vulgare (Wheat) germ oil, Virola surinamensis (Ucuuba) seed butter, Vitis vinifera (Grapeseed) oil, Zea mays (Corn) oil and mixtures thereof.

It is more preferred that the lipophilic material is a wax selected from 12-hydroxystearic acid, glycerol tristearate, glyceryl tridodecanoat, glycerol cinnamate, carnauba wax, candelilla wax, beeswax, permulgin wax, rice bran wax, siliconyl candelilla wax, rhuswax, sugarcane wax, tribehenin, C₁₈₋₃₆ acid triglyceride, C₁₈₋₃₆ acid glycol ester, cetyl alcohol, stearyl alcohol, behenyl alcohol, polyethylene, or a mixture thereof. In one embodiment, the lipophilic material is selected from hydrogenated soybean oil, hydrogenated palm oil, hydrogenated peanut oil, hydrogenated cottonseed oil, beeswax, C₁₈₋₃₆ acid triglyceride, tribehenin, cetyl alcohol, stearyl alcohol, behenyl alcohol, polyethylene, shea butter, coconut oil, or a mixture thereof. In another embodiment, the lipophilic material is 12-hydroxystearic acid, hydrogenated castor oil, C₁₈₋₃₆ acid triglyceride, tribehenin, cetyl alcohol, stearyl alcohol, behenyl alcohol, or a mixture thereof.

The personal care composition may comprise from 1 to 30%, or 5 to 25%, or 10 to 22%, or 12 to 20%, or 14 to 22%, or 13 to 20%, or 13 to 18% lipophilic material by weight of the composition.

The personal care compositions disclosed herein may also optionally comprise at least one powder. Illustrative but nonlimiting powders include mineral powder (e.g., talc, magnesium silicate, mica, sericite, silica, magnesium hydroxide, synthetic fluorphlogopite, calcium silicate, calcium carbonate, calcium hydroxide, aluminum silicate, kaolin, bentonite, and montmorillonite); an organic powder (e.g., polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride); a starch (e.g., Maranta arundinacea (Arrowroot) powder, Manihot esculenta (Cassava) starch, Manihot esculenta root (Tapioca) starch, Zea mays (Corn) starch, Oryza sativa (Rice) starch, Solanum tuberosum (Potato) starch, Triticum vulgare (Wheat) starch, Cycas revoluta (Sago) starch, Hordeum vulgare (Barley) starch, sodium hydroxypropyl starch phosphate and aluminum starch, octenylsuccinate or mixtures thereof) that may be native, modified, or a mixture thereof; or a combination thereof. Powders may be incorporated in the disclosed personal care composition in amounts of 0.01 to 10%, or 0.1 to 9%, or 1 to 8%, or 2 to 7% by weight of the composition. In one embodiment, the personal care composition comprises talc, corn starch, tapioca starch, rich starch, or mixtures thereof. In another embodiment, the personal care composition may be substantially free of talc. In yet another embodiment, the personal care composition may comprise no (i.e., 0% by weight) talc.

Preferably, the disclosed personal compositions are substantially free of added alcohol (e.g., excluding alcohol residual from the manufacturing or extraction processes of a raw material or blend of raw materials). It is particularly preferred that the personal care compositions have less than 1%, more preferably, 0.1%, and, most preferably, 0% alcohol by weight of the composition. “Alcohol,” as used herein, consists essentially of methyl alcohol (methanol), ethyl alcohol (ethanol), isopropyl alcohol, alcohol denatured or specifically denatured (SD) alcohol.

The disclosed personal care composition can comprise other optional components. The CTFA Personal Care Ingredient Handbook, Second Edition, 1992, which is incorporated by reference herein in its entirety, describes a wide variety of non-limiting personal care and pharmaceutical ingredients commonly used in the personal care industry. Illustrative examples include but are not limited to antioxidants, astringents, binders, biological additives, buffering agents, chelators, colorants/dyes, conditioners, essential oils, exfoliating agents, fragrances, natural extracts, pH adjusters, polymers, preservatives, sensory modifiers, rinse-/wash-off aids, skin appearance modifiers (e.g., skin-lightening agents and skin-smoothing agents), solvent, surfactant, suspending agent, viscosity modifiers, and vitamins. Such an optional component may be included from 0.001% to 10%.

It is also within the scope of the disclosed personal care compositions to further include one or more antioxidants, which may be natural, naturally derived, or synthetic antioxidants. Antioxidants may be any antioxidant desirable for use in personal care compositions, provided such antioxidants are compatible for use in substantially anhydrous compositions. Illustrative but nonlimiting examples of such antioxidants comprise alpha hydroxy acids (e.g., citric acid, lactic acid, glycolic acid and mandelic acid), amino acids and derivatives thereof, beta-hydroxy acids (e.g., propanoic acid and salicylic acid), butylated hydroxytoluene (BHT), carotenes, carotenoids, co-enzyme Q10, didodecyl 3,3′ thiodipropionate, glutathione, imidazole and derivatives thereof, octadecyl di-t-butyl-4-hydroxyhydrocinnamate, pentaerythrityl tetra-di-t-butyl hydroxyhydrocinnamate, peptides and derivatives thereof, plant extracts (e.g., Rosmarinus officinalis (rosemary) leaf extract and Camellia sinesis (green tea) leaf extract), polyhydroxy acids (e.g., gluconic acid, gluconolactone and lactobionic acid), polyphenols (e.g., anthocyanin, ellagic acid, flavonoid, tannin), and resveratrol. Desirable antioxidants, when used, may collectively make up from 0.001 to 12%, or 0.01 to 5%, or 0.02 to 3% by weight of the personal care composition.

Another class that may be used in the personal care composition as antioxidants includes vitamins. Illustrative vitamins include, but are not limited to, vitamin B₁, vitamin B₂, vitamin B₃, vitamin B₅ (panthenol), vitamin B₆, vitamin C, vitamin E, vitamin F, vitamin K, folic acid, and biotin. Derivatives of the vitamins may also be employed. For instance, derivatives of vitamin E include tocopheryl acetate, tocopheryl palmitate and tocopheryl linoleate. It is preferred that the antioxidant comprises vitamin E and derivatives thereof.

Fragrance is also a desirable optional component. The term “fragrance” is defined herein as odoriferous compounds or a mixture of odoriferous compounds, optionally mixed with a desirable solvent diluent or carrier, which is employed to impart a desired odor. A perfumery molecule or a group of perfumery molecules formulated as a perfume is generally incorporated in most personal care compositions. Any perfumery molecule/fragrance which is cosmetically acceptable may be included in the disclosed personal care composition disclosed herein. Desirable materials include conventional perfumes, such as perfume oils and also include so-called deo-perfumes, as described in EP 545,556 and other publications. Highly preferred fragrance components used in personal care compositions are cyclic and acyclic terpenes and terpenoids. These materials are based upon isoprene repeating units. Examples include alpha and beta pinene, myrcene, geranyl alcohol and acetate, camphene, dl-limonene, alpha and beta phellandrene, tricyclene, terpinolene, allocimmane, geraniol, nerol, linanool, dihydrolinanool, citral, ionone, methyl ionone, citronellol, citronellal, alpha terpineol, beta terpineol, alpha fenchol, borneol, isoborneol, camphor, terpinen-1-ol, terpin-4-ol, thymol, dihydroterpineol, methyl chavicol, anethole, 1,4- and 1,8-cineole, geranyl nitrile, isobornyl acetate, linalyl acetate, caryophyllene, alpha cedrene, guaiol, patchouli alcohol, alpha and beta santalol, and mixtures thereof. Levels of incorporation are preferably up to 4%, particularly from 0.1% to 3%, and especially from 0.7% to 2%. It is also within the scope of the invention that the composition is substantially free of fragrance. In one embodiment, the composition comprises no (i.e., 0% by weight) fragrance.

Other additional optional skin benefit agents desirable for use in the disclosed personal care composition include minerals and skin nutrients such as magnesium, calcium, copper, zinc and other metallic components; retinoic acid and its derivatives (e.g., cis and trans); retinal; retinol; retinyl esters such as retinyl acetate, retinyl palmitate, and retinyl propionate; resorcinol and resorcinol derivatives (particularly 4-substituted resorcinol derivatives, including 4-isopropyl resorcinol, 4-hexyl resorcinol, 4-cyclopentyl resorcinol, thiamidol, 4-cyclohexyl resorcinol and acylated forms thereof); saccharide; saccharide isomerate, ceramides (e.g., Ceramide 1, Ceramide 3, Ceramide 3B and Ceramide 6) and pseudoceramides, 12-hydroxystearic acid, petroselinic acid, conjugated linoleic acid, and octadecanoic acid. Such skin benefit agents, when used, collectively make up from 0.001 to 12% by weight of the personal care composition.

A wide selection of botanical extracts may optionally be included in the personal care compositions disclosed. The extracts may either be soluble in water or oil, carried in a solvent that is hydrophilic or hydrophobic, respectively, provided that they are compatible with a personal care composition that is substantially anhydrous. Illustrative examples include those extracted from green tea, yarrow, chamomile, licorice, aloe vera, citrus unshui, willow bark, hops, alfalfa, algae, jojoba, witch hazel, sage, thyme, Marsdenia Condurango bark, and rosemary, as well as oils such as those derived from sea buckthorn, moringa, argan, avocado, calendula, algal, eucalyptus, lavender, bergamot, and marula. Soy extracts may be used and especially when it is desirable to include retinol. Such extracts, when used, are preferably employed in collective amounts ranging from 0.001 to 12% by weight of the personal care composition.

Another optional component desirable for use includes hemp oil with 2.5 to 25% by weight cannabigerol and/or cannabidiol at from 0.5 to 10 percent by weight. When used, such oil makes up from 0.0001 to 12% by weight of the composition, and preferably, from 0.01 to 5% by weight of the personal care composition.

Personal care chemists are familiar with appropriate preservatives and routinely choose them to satisfy preservative tests and product stability tests. Preservative systems should be selected having regard for the use of the composition and possible incompatibilities between the preservatives and other ingredients in the formulation. Exemplary examples of preservatives for use in substantially anhydrous compositions include, without limitations, butyl paraben, ethyl paraben, methyl paraben, and propyl paraben. Preservatives are preferably employed in amounts ranging from 0.01 to 2% by weight of the personal care composition. It is particularly preferred that the personal care composition is substantially free of preservatives, or, more preferably, is preservative-free. In one aspect, the personal care composition may be paraben-free.

The personal care compositions disclosed herein may have a product or stick hardness from 60 to 110 mm*10, as measured on a penetrometer by use of the penetration test (ASTM D-5, D1321, D217) with an ASTM D-1321 needle. In some embodiments, the average product hardness may be from 62 to 100 mm*10, and in others from 65 to 95 mm*10, and in still others from 65 to 90 mm*10. With regards to results, the units for penetration are in tenths of a millimeter (i.e., 1/10 mm=100 microns). Thus, for example, 60 units equals 6 mm, or 60 mm*10. An average hardness measurement was obtained by averaging the results of 6 total measurements for each the top and the side of the product.

The personal care compositions disclosed herein may also have a product or stick payout ranging from 0.15 to 0.25 grams (g) on average. “Payout,” as used herein, refers to the amount of the composition, as measured in grams (g), deposited or spread onto the user's skin or a substrate surface from the personal care stick during dispensing/topical application. “Parity payout” as used herein, refers to the comparable payout of the personal care composition made according to this invention versus other reference compositions. In one embodiment, the personal care composition gives parity payout during topical application. Payout was measured by having trained panelists apply three (3) up and down strokes of each composition in a stick format to the underarm and measuring the dosage in grams (g). It has unexpectedly been found that when compared to reference compositions without reduced volatile silicone content, the personal care compositions disclosed herein produced payout on par with that of the reference compositions.

In addition, it has also surprisingly been found that the disclosed personal care compositions may provide a parity sensory feel at application and throughout the day. “Improved sensory feel,” as used herein, refers to a reduced sticky, tacky, and/or greasy sensorial experience upon topical application of the composition and during the entire duration of use. The term parity means that a certain quality of the disclosed personal care composition matched or was comparable to that same quality in a non-reduced volatile silicone reference composition. This was unexpected because volatile silicones evaporate quickly and provide a dry feel for a better sensorial experience upon application, so a reduction of such volatile silicones was expected to negatively impact sensory. However, it was found that the personal care compositions disclosed herein resulted in parity sensory as the non-reduced volatile silicone reference compositions.

In another aspect of the personal care compositions disclosed herein, there is provided a method of attaining antiperspirant benefits comprising the steps of: (a) identifying skin in need of antiperspirancy and/or deodorant, preferably, the axillae; and (b) topically applying, directly or indirectly, to the skin a composition made according to the first aspect of the compositions disclosed herein.

In yet another aspect, there is provided a use of the disclosed personal care composition for delivering an antiperspirant and/or deodorant benefit.

In still another aspect, there is provided a method of manufacturing the disclosed personal care composition.

Many types of packaging can be used to store and deliver the disclosed personal care composition. The selection of packaging is dependent upon the personal care end-use and the physical properties of the composition itself. The personal care product is in the form of a stick; preferably, comprising the composition as hereinbefore described comprised within a stick dispenser product package. Usual stick products are packaged so that there is a plastic dome on top of the stick, between the solid stick formulation and the cap. The dome is removed and typically discarded by the consumer prior to first use. There is also provided a cap which can be replaced following each use. Preferably, the packaging is sustainable and comprises PCR (post-consumer recycled or post-consumer resin) plastic, or consists essentially of PCR plastic. It is also preferable that the packaging is suitable for refilling or is suitable for packaging a refill personal care composition.

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above. The term comprises is also meant to encompass the terms consisting essentially of and consisting of. For the avoidance of doubt, a composition comprising at least one non-volatile emollient oil having a boiling point of at least 215° C. at atmospheric pressure, a lipophilic material, and less than 25% antiperspirant active by weight of the composition, wherein the composition has reduced volatile silicones, and further wherein the composition is substantially free of emulsifier is meant to include a composition consisting essentially of the same and a composition consisting of the same. As to the percentages used herein, the same are meant to be by weight of ingredient (e.g., not including any water it may be supplied with), unless noted otherwise.

By an “antiperspirant composition” as used herein, is meant to include a composition for topical application to the skin of mammals, especially humans. Such a composition is preferably of the leave-on type. By a leave-on composition is meant a composition that is applied to the desired skin surface and left on for one minute to 24 hours after which it may be wiped or rinsed off with water, usually during the regular course of personal washing. The composition may also be formulated into a product which is applied to a human body for improving the appearance, cleansing, odor control or general aesthetics. The composition of the present invention can be in the form of a liquid, lotion, cream, gel or stick form and may be delivered through a roll-on device or using an aerosol can which contains a propellant.

The term “skin” as used herein includes the skin on the face, neck, chest, back, arms, axilla, buttocks, hands, legs and scalp. Skin benefit agent, as used herein, is meant to include a component that improves a facial or body characteristic after topical application like a skin characteristic and/or benefits the same wherein the skin benefit agent can be incorporated in a desirable format. The term “derivative(s)” as used with reference to ingredients herein refers to compounds which may be formed from a precursor compound and/or is a structural or chemical analog. All states of matter, as used herein, such as solid, liquid and gas relate to the specified state of matter at 25° C. and atmospheric pressure. The term “balance” as used herein means a sufficient quantity remaining to reach 100% by weight of the composition. Substantially free of, as used herein, is intended to mean comprising less than 2%, preferably, less than 1% by weight, more preferably, less than 0.5% by weight, and, most preferably, 0% by weight. Anhydrous, as used herein, is intended to mean comprising 0% by weight of any added water. Ambient temperature, as used herein, refers to a temperature of 20 to 25° C. When referring to oil solubility and/or water solubility (e.g., “water-soluble” or “oil-soluble”), the conditions are ambient temperature and atmospheric pressure. “Melting point” refers to the temperature at which the solid and liquid forms of a pure substance can exist in equilibrium. “Melting point” for the purpose of the present invention is specified at a standard pressure such as 1 atmosphere (atm).

The following Examples are provided to further illustrate an understanding of the invention. The Examples are not intended to limit the scope of the claims. One of ordinary skill in the art will recognize that variations of the method steps that differ from the examples given may be practiced without deviating from the teachings of the present compositions.

### Examples

All samples were made by mixing the mentioned ingredients under conditions of moderate sheer, 22° C. to 95° C., and atmospheric pressure.

Example 1: Antiperspirant compositions

| | Sample | | | |
|---|---|---|---|---|
| Ingredient | 1 | A | B | 2 |
| Cyclopentasiloxane >96% active | 32.75 | 15.25 | 13.15 | 25.15 |
| Isopropyl Palmitate | — | 11.00 | 16.50 | 16.50 |
| PPG-14 Butyl Ether | 17.50 | 17.50 | 10.10 | — |
| Mineral Oil 125-135 SUS | — | 5.50 | 10.00 | 10.00 |
| Stearyl Alcohol | 17.50 | 17.50 | 15.50 | 13.60 |
| Hydrogenated Castor Oil | 2.50 | 2.50 | 3.40 | 3.40 |
| Astringent metal salt | 24.00 | 24.00 | 24.00 | 24.00 |
| (antiperspirant active) | | | | |
| PEG-8 Distearate | 2.00 | 2.00 | — | — |
| Steareth-100 | — | — | 0.30 | 0.30 |
| Mineral powder | Balance | Balance | Balance | Balance |
| Antioxidant | 0.05 | 0.05 | 0.05 | 0.05 |
| Fragrance | 1.70 | 1.70 | 1.00 | 1.00 |

Antiperspirant stick compositions were made by combining the above ingredients in accordance with the personal care compositions disclosed herein in samples A and B. Samples 1 and 2 were made by combining the above ingredients not in accordance with the disclosed personal care compositions and serve as the non-reduced volatile silicone reference compositions for samples A and B, respectively. Samples A and B comprise a fifty-percent reduction in inclusion level for volatile silicone (cyclopentasiloxane) when compared to samples 1 and 2.

### Example 2: Mean Measurements or Scores of Attributes

A series of clinical experiments were conducted to evaluate personal care compositions as disclosed herein (samples A and B) against those out-of-scope of the disclosed personal care compositions (samples 1 and 2). The clinical involved trained panelists assessing a variety of attributes known to be of consumer importance. Assessments may then be made at various time points during and after application. Perceived drying time was then evaluated by the panelists.

| | Sample | | | |
|---|---|---|---|---|
| Mean measurements | 1 | A | B | 2 |
| Payout (mean dosage) (g) | 0.17 | 0.20 | 0.21 | 0.21 |
| Hardness (from top) (mm*10) | 71.90 | 71.70 | 70.30 | 72.20 |
| Hardness (from side) (mm*10) | 73.50 | 73.20 | 73.40 | 73.20 |
| Perceived drying time (mins) | 4.00 | 5.00 | 5.00 | 5.00 |
| Actual drying time (mins) | 6.00 | 6.00 | 6.00 | 7.00 |
| Mean scores of attributes | | | | |
| Glide (during application) | 46.00 | 52.00 | 51.00 | 58.00 |
| Smoothness | 61.00 | 61.00 | 63.00 | 67.00 |
| (during application) | | | | |
| Wetness (immediately | 8.00 | 10.00 | 9.00 | 10.00 |
| after application) | | | | |
| Wetness (8 mins | 3.00 | 4.00 | 4.00 | 4.00 |
| after application) | | | | |
| Stickiness (immediately | 4.00 | 5.00 | 5.00 | 5.00 |
| after application) | | | | |
| Stickiness (8 mins | 2.00 | 3.00 | 3.00 | 3.00 |
| after application) | | | | |
| Visibility (immediately | 11.00 | 10.00 | 10.00 | 14.00 |
| after application) | | | | |
| Visibility (10 mins | 6.00 | 7.00 | 6.00 | 10.00 |
| after application) | | | | |
| Greasiness (10 mins | 10.00 | 16.00 | 14.00 | 18.00 |
| after application) | | | | |

Comparison of two compositions made according to the personal care compositions disclosed herein (samples A and B) with reference compositions containing approximately 50% more volatile silicone (samples 1 and 2) were made in terms of objective measurements such as hardness and payout. A study of drying time, glide, smoothness, and stickiness during and post-application to the axilla area was also conducted. Results demonstrate that, across all measurements and attributes, there was no significant difference between the reduced volatile silicone compositions when compared to their respective reference compositions. Comparable results were achieved between the present personal care compositions and their non-reduced volatile silicone counterparts.

The results are unexpected because volatile silicones evaporate quickly and provide a dry feel for a better sensorial experience upon application, so a reduction of such volatile silicones was expected to negatively impact sensory. Moreover, it was expected that non-volatile silicones, by virtue of being not as volatile as volatile silicones, thereby staying on the surface to which the product was applied longer, would have contributed to, for example, additional greasiness and wetness as well as a longer perceived and actual drying time. However, it was found that the personal care compositions disclosed herein resulted in parity sensory as the non-reduced volatile silicone reference compositions.

1. A personal care composition comprising:
a) at least one non-volatile emollient oil having a boiling point of at least 215° C. at atmospheric pressure,
b) 1 to 30% a lipophilic material by weight of the composition, and
c) less than 25% antiperspirant active by weight of the composition,
wherein the composition comprises less than 30% by weight silicone,
wherein the antiperspirant active is an astringent metal salt,
and further wherein the composition is substantially free of water and emulsifier.

2. The personal care composition of claim 1, wherein the non-volatile emollient oil comprises mineral oil, PPG-14 butyl ether, isopropyl isostearate, isostearyl isostearate, isopropyl myristate, isopropyl palmitate, petrolatum, butyl stearate, cetyl octanoate, butyl myristate, myristyl myristate, C₁₂₋₁₅ alkylbenzoate, octyldodecanol, isostearyl isostearate, octododecyl benzoate, isostearyl lactate, isostearyl palmitate, isobutyl stearate, dimethicone, dimethicone copolyol, PEG-12 dimethicone, polydecene, or mixtures thereof.

3. The personal care composition of claim 1, wherein the non-volatile emollient oil has a flash point of at least 80° C.

4. The personal care composition of claim 1, wherein the non-volatile emollient oil has a boiling point ranging from 215° C. to 400° C.

5. The personal care composition of claim 1, wherein the composition comprises 15 to 45% by total weight non-volatile emollient oil of the composition.

6. The personal care composition of claim 1, wherein the composition comprises 25 to 40% by total weight non-volatile emollient oil of the composition.

7. The personal care composition of claim 1, wherein the lipophilic material comprises hydrogenated oil, a fatty alcohol, a fatty acid, a fatty ester, a petroleum oil, a wax, or mixtures thereof.

8. The personal care composition of claim 1, wherein the lipophilic material comprises 12-hydroxystearic acid, glycerol tristearate, glyceryl tridodecanoat, glycerol cinnamate, carnauba wax, candelilla wax, beeswax, permulgin wax, rice bran wax, siliconyl candelilla wax, rhuswax, sugarcane wax, tribehenin, C₁₈₋₃₆ acid triglyceride, C₁₈₋₃₆ acid glycol ester, cetyl alcohol, stearyl alcohol, behenyl alcohol, polyethylene, or mixtures thereof.

9. The personal care composition of claim 1, wherein the composition comprises 5 to 25% of the lipophilic material by weight of the composition.

10. The personal care composition of claim 1, wherein the composition comprises 0.1 to 24.5% antiperspirant active by weight of the composition.

11. The personal care composition of claim 1, wherein the antiperspirant active is aluminum chlorohydrate, aluminum zirconium tetrachlorohydrex gly, aluminium sesquichlorohydrate of chemical formula Al₂OH_{4.4}Cl_{1.6} to Al₂OH_{4.9}Cl_{1.1} also comprising water-soluble calcium salt and amino acid, or a mixture thereof.

12. The personal care composition of claim 1, wherein the silicone is a volatile cyclic polydimethylsiloxane having three to seven silicon atoms.

13. The personal care composition of claim 1, wherein the volatile silicone is cyclotetrasiloxane, cyclopentasiloxane, cyclohexasiloxane, or mixtures thereof.

14. The personal care composition of claim 1, wherein the composition comprises 1 to 30% volatile silicone by weight of the composition.

15. The personal care composition of claim 1, wherein the composition comprises 2 to 17% volatile silicone by weight of the composition.

16. The personal care composition of claim 1, wherein the composition is substantially free of humectant selected from the group consisting of glycerin, PEG-4, or mixtures thereof.

17. The personal care composition of claim 1, wherein the composition is substantially free of emulsifier.

18. The personal care composition of claim 1, wherein the composition has a product or stick hardness ranging from 60 to 110 mm*10 when measured on a penetrometer.

19. The personal care composition of claim 1, wherein the composition has a product or stick hardness ranging from 62 to 100 mm*10 when measured on a penetrometer.

20. The personal care composition of claim 1, wherein the composition further comprises at least one of thymol, terpineol, resorcinol and derivatives thereof, antioxidant, vitamin B₃, retinyl ester, 12-hydroxystearic acid, stearic acid, palmitic acid, fatty alcohol, saccharide, cholic acid derivative, glycerol monolaurate, chitosan or a salt thereof, zinc-based deodorizing agent, titanium compound chelated by alkanolamine with an acid and polyhydric alcohol, or mixtures thereof.
